(19) United States
(12) Patent Application Publication
(10) Pub. No.: US 2005/0065496 A1
(43) Pub. Date: Mar. 24, 2005

(54) Chemiluminescently illuminated suction appliances

(76) Inventors: James S. Simon, Tiburon, CA (US); Robert A. Simon, Tiburon, CA (US)

Correspondence Address: MORRISON & FOERSTER LLP, 755 PAGE MILL RD, PALO ALTO, CA, 94304-1018, US

(21) Appl. No.: 10/936,341

(22) Filed: Sep. 7, 2004

Related U.S. Application Data
(63) Continuation of application No. PCT/US03/06868, filed on Mar. 6, 2003.
(60) Provisional application No. 60/416,079, filed on Oct. 4, 2002.
(60) Provisional application No. 60/362,492, filed on Mar. 6, 2002.

(51) Int. Cl. A61M 31/00

(52) U.S. Cl. 604/500

(57) ABSTRACT

This invention relates to chemiluminescent suction devices and methods for using the devices. The devices may be useful as medical, industrial, and consumer products. They may be disposable and may employ an integrated or attachable chemiluminescent source. In some variations, the device may include a supporting structure or body that is at least partially translucent or transparent for transmitting chemiluminescent illumination.

### CROSS REFERENCE TO RELATED APPLICATIONS

This application is a continuation application of PCT/US03/06868, filed Mar. 6, 2003, which claims benefit of U.S. Provisional Patent Application No. 60/416,079, filed Oct. 4, 2002 and U.S. Provisional Patent Application No. 60/362,492, filed Mar. 6, 2002, which are hereby incorporated by reference in their entirety.

### FIELD OF INVENTION

This invention relates to devices that may be chemiluminescently illuminated and useful as medical, industrial, and consumer products. They may be disposable and may employ an integrated or attachable chemiluminescent source. In some variations, the device may include a supporting structure or body that is at least partially translucent or transparent for transmitting chemiluminescent illumination.

### BACKGROUND

Safe and efficient use of many medical and dental appliances requires illumination. The illumination is desirably, amongst other things, precise, convenient, and not overly cumbersome or cost prohibitive.

A conventional approach to provide illumination involves the use of ambient lighting. Overhead lights may provide the ambient or room light. Although ambient lighting may provide adequate illumination for the surgeon in certain situations, ambient lighting is not always available as an illumination option such as in the military and paramedic (emergency) environments. Additionally, certain diagnostic procedures (e.g., radiology) preclude the use of ambient lighting because these procedures (e.g., fluoroscopy) are performed in a dark room or in lowered ambient lighting. Such procedures are hindered by direct lighting.

Another approach to providing illumination involves the use of additional attendants to direct a light source at the surgical field as the medical practitioner performs the procedure. The use of additional attendants to hold the light frees the medic's hands and provides adequate illumination. However, a shortcoming of this technique is that additional attendants may not always be available and, even if the attendants are available, their presence increases the cost of the procedure.

Internally lighted devices provide solutions to some of the above identified problems. Internally lighted devices also lessen improper direction or reflection of certain light sources such as overhead lamps. Medical and dental appliances have previously been lighted primarily by fiberoptic illuminators that project light through an attached cable containing glass or plastic fibers; the cable is typically connected to a surgical instrument or headlight device that projects light onto the area of interest.

Although use of fiber optics in medical appliances has reduced some of the above mentioned problems, fiber optically illuminated medical devices still require an external power or light source. Fiber optically illuminated medical devices are thus not conveniently functional in many situations such as emergency or military scenarios.

Proper placement of medical devices during a procedure can be critical. For example, proper endotracheal-device placement is critical in patients requiring assured airway access for breathing, anaesthetic, or resuscitative purposes. During intubation, a clear airway is critical because the airway can become obstructed by foreign bodies, fluid, or tissues (such as the patients tongue or regions of swelling). Lighting during intubation procedures is often inadequate and airway obstructions may compromise visualization of the proper anatomical pathways.

Chemiluminescently illuminated medical and dental appliances are useful without external power sources, may be used under extreme situations (during power failures, emergency surgeries, or on a military field), and may easily be designed for disposability (to eliminate the costs of sanitization and the risks of repeated use). Depending upon the application and structure of the medical appliance, the region of chemiluminescence may be integrated into the overall structure of the medical appliance or may be a separate section that is attached to the original structure of the medical appliance. The integration of the chemiluminescent material into disposable medical appliances may serve as improvements over certain medical appliances to which the chemiluminescence was attached for a number of reasons: (1) there may be fewer components to manipulate, desirable during emergency situations; (2) integrated components may reduce the amount of material later to be disposed; (3) an entirely disposable unit may reduce sterilization costs and errors; (4) indented or grooved attachment regions may provide incubation sites for contamination.

An apparatus for endoscopic examination of a body cavity using chemiluminescent light source is described in U.S. Pat. Nos. 5,179,938 and 5,329,938, both to Lonky. The described apparatus has a chemiluminescent light source having particular wavelength characteristics made up of blue and green wavelength. The device may be for improved screening of the vaginal cavity.

The regions of chemiluminescence used in the invention described herein may be in the form of separate chemiluminescent components that are attachable to the main structure of the medical appliance (via connectors, grooves, or cavities, etc. in the main structure). The chemiluminescent regions or sources of this invention may also be integrated into a region of (or into the whole of) the appliance depending of course upon the area to be illuminated. The chemiluminescent regions may have containment walls that are flexible or stiff.

Proper illumination may be even more critical in situations where a surgeon or paramedic providing medical intervention needs to make an incision on the body of the patient. The medical professional relies on visualization in order to perform the procedure, and a properly positioned light source ensures that there is adequate contrast and illumination for proper execution of the procedure. Not having adequate illumination not only could lead to failure of the procedure but could also result in irreversible injury to the patient.

Thus, in situations where surgical interventions are to be provided in environments having poor illumination such as on a battlefield or at a scene of a car accident, a well positioned light source that is lightweight and independent of an external power supply is desirable.

### SUMMARY OF THE INVENTION

The present invention involves chemiluminescently illuminated devices. They typically include an operational portion directed or configured for a particular application and an integrated or separately attachable chemiluminescent light source. The light source may surround (or be contained within) a portion of the device. Also, the phrase “light source” is intended to refer to at least one light source.

The devices may be configured to employ previously known operational portion or portions of a selected appliance. They may be configured to emit light onto those regions of the body, where the device is a medical device, and illuminate the field of the procedure for which the appliance is intended. The appliance may comprise an at least partially translucent medical or dental appliance that may be introduced into or engaged with the human body. The words “translucence” or “translucent” are intended to describe the ability to permit the passage of at least visible light but may refer to that ability whether the transmission is of diffuse light (translucent) or of transmission of light without substantial diffusion (transparent). At least a portion of the inventive devices may comprise materials that are translucent. These materials may be polymeric. The invention also includes methods of using the described devices and of illuminating an intended procedure, operation, or use of the device whether the device is translucent, semi translucent, or not.

The chemiluminescent material may be of the types that are induced or catalyzed (similar to that sold in Cyalume “light sticks”). Chemiluminescent materials are well known and appropriate ones are readily available from commercial sources. The various materials of construction should be compatible for use within the human body. The devices may be designed and adapted in such ways that they are either disposable or re-useable or portions of the devices are disposable or re-useable.

The chemiluminescent regions of this invention may be integrated into the device itself, e.g., by placement of an open volume suitable for containing the chemiluminescent liquid, gel, or solid during manufacture of the device. Alternatively, they may be independent and placed upon or in the device after manufacture of the basic device in such a way that they are fixed, e.g., via an adhesive, by solvent welding, by melting etc., or by mechanical fasteners or in some other fashion. The region may be adapted to be removable. One commercially available method for producing chemiluminescence is via the use of a separate volume that is breakable or rupturable, separating a catalyst or reactant from a chemiluminescent precursor. Other ways of maintaining separation prior to use, e.g., valves and seals, are also suitable.

Examples of the devices include: forceps, anoscopes, intubation devices, trocars, dental suction devices, surgical suction devices, tongue depressors, intravenous bag apparatus, surgical headlamp, surgical retractors, sternum retractors, catheter tips, intravenous needle tips, otoscope specula, butterfly needle, dental mirrors, tracheostomy devices, vaginal specula, laryngeal masks, laryngoscopes, and laryngeal intubation devices (blades and handles).

In another aspect of the invention, regions of a chemiluminescence light source may be in the form of separate structures that are attachable to the main structure of the medical device (via connectors, grooves in the structure into which the illumination structures fit, etc.). The regions or sources may be integrated into a region of or into the whole of the appliance depending of course upon the area to be illuminated. The regions may have containment walls that are flexible or stiff.

### BRIEF DESCRIPTION OF THE DRAWINGS

FIGS. 7A, 7B, and 7C are, respectively, a perspective view and two cross-sectional views of an illuminated dental suction device made according to this invention.

FIGS. 8A and 8B are perspective views of illuminated surgical suction devices made according to this invention.

FIG. 8C is a cross sectional view of the device shown in FIG. 8B taken along 8C-8C.

### DESCRIPTION OF THE INVENTION

Medical appliances are described herein. The medical appliances may be directed and configured for various uses and procedures and have particular suitability where self illumination is advantageous such as in emergency and military environments. The described devices typically comprise a chemiluminescent source that provides illumination. The chemiluminescent source or region, as will be explained in more detail below, is variously coupled to, associated with, or incorporated into the device.

The chemiluminescent materials may be comprised of various chemicals that are well known to one skilled in the art to be able to provide luminescence when reacted with one another. A chemical luminescent reaction requires two or more chemicals to react with one another. When the reaction occurs, the electrons in the new product get excited, and they emit light when the electrons fall back into their normal orbits. Examples of chemiluminescent materials are disclosed in the following patents: U.S. Pat. No. 3,539,794 issued Nov. 10, 1970 to Rauhut et al.; U.S. Pat. No. 3,350,553 issued Oct. 31, 1967 to Cline; U.S. Pat. No. 3,729,425 issued Apr. 24, 1973 to Andress et al.; U.S. Pat. No. 3,808,414 issued Apr. 30, 1974 to Roberts; U.S. Pat. No. 3,893,938 issued Jul. 8, 1975 to Rauhut; U.S. Pat. No. 4,814,949 issued Mar. 21, 1989 to Elliott; U.S. Pat. No. 4,635,166 issued Jan. 6, 1987 to Cameron; U.S. Pat. No. 5,348,690 issued Sep. 20, 1994 to Cohen et al.; U.S. Pat. No. 5,488,544 issued Jan. 30, 1996 to Ladyjensky; and U.S. Pat. No. 5,552,968 issued Sep. 3, 1996 to Ladyjensky; each of which is incorporated herein by reference in its entirety.

One combination of chemiluminescent materials commonly used in the industry comprises of phenyl oxalate ester, fluorescent dye solution and hydrogen peroxide. The fluorescent dye may be a diphenyl-anthracene, a bis(phenylethynyl) anthracene, or a tetraphenylnaphthacene. The hydrogen peroxide may be separately contained in a subcompartment and isolated from the phenyl oxalate ester and the fluorescent dye. When the user intends to activate the chemiluminescent light source, the integrity of the subcompartment is compromised allowing mixing of the organic oxalate (R—O—CO—CO—O—R, wherein Rs stand for organic groups) with the hydrogen peroxide, H₂O₂. The product loses energy to relax back down to its ground state by dumping energy to the fluorescent dye molecule. The dye releases the energy as brilliantly colored light. The subcompartment may comprise a glass ampule containing the hydrogen peroxide. The ampule may be located within a main chamber containing the Oxalate Ester and fluorescent dye solution. Other chemiluminescent agents that are well known to one skilled in the art may also be implemented as the light source for the chemiluminescently illuminated device.

Dental Suction Appliance

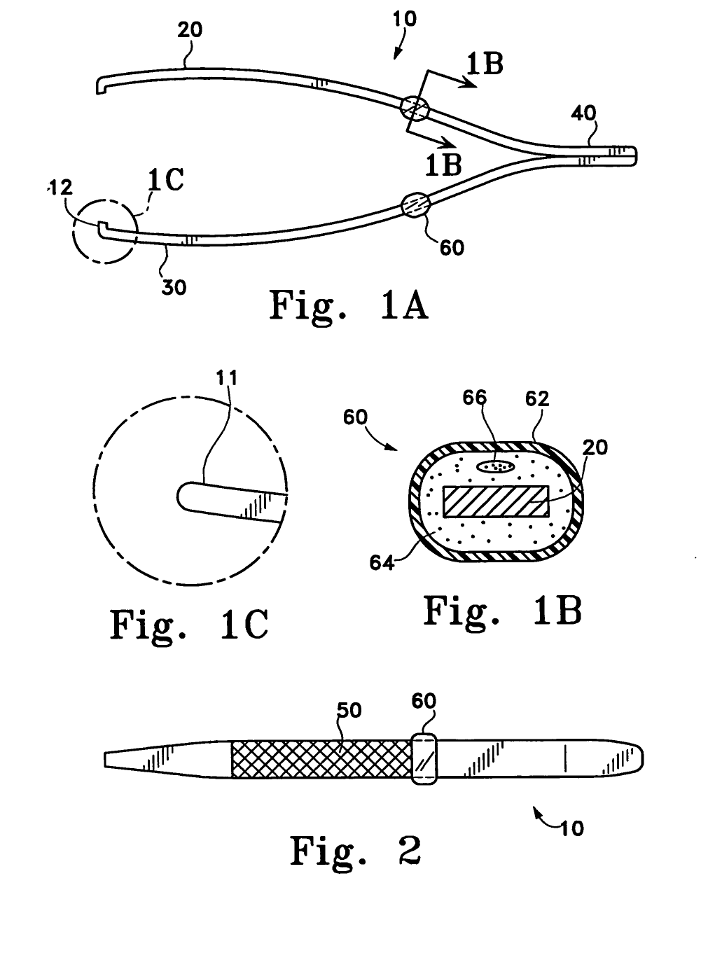

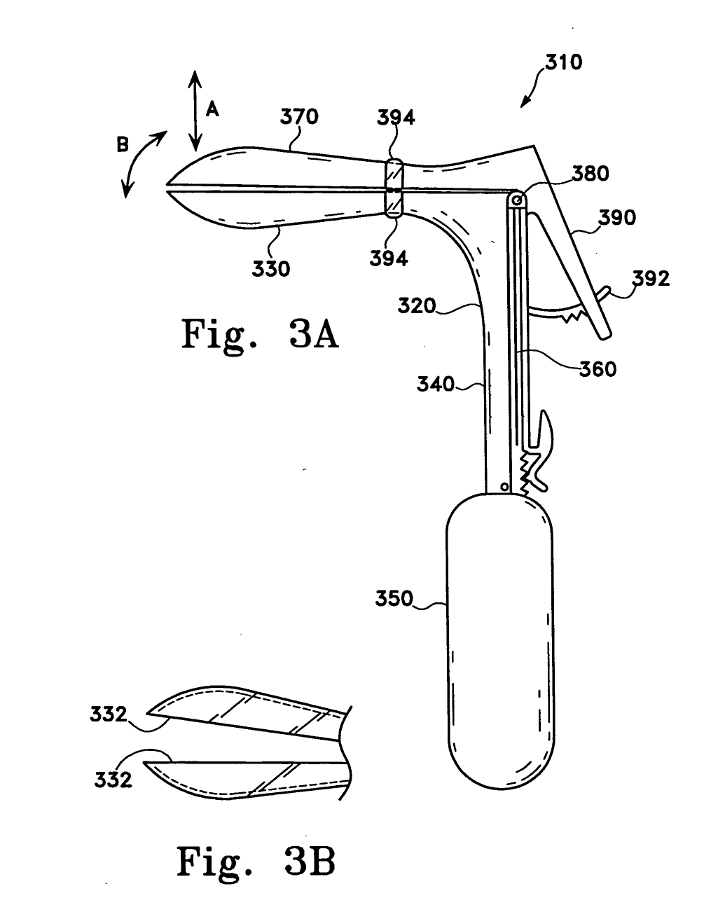

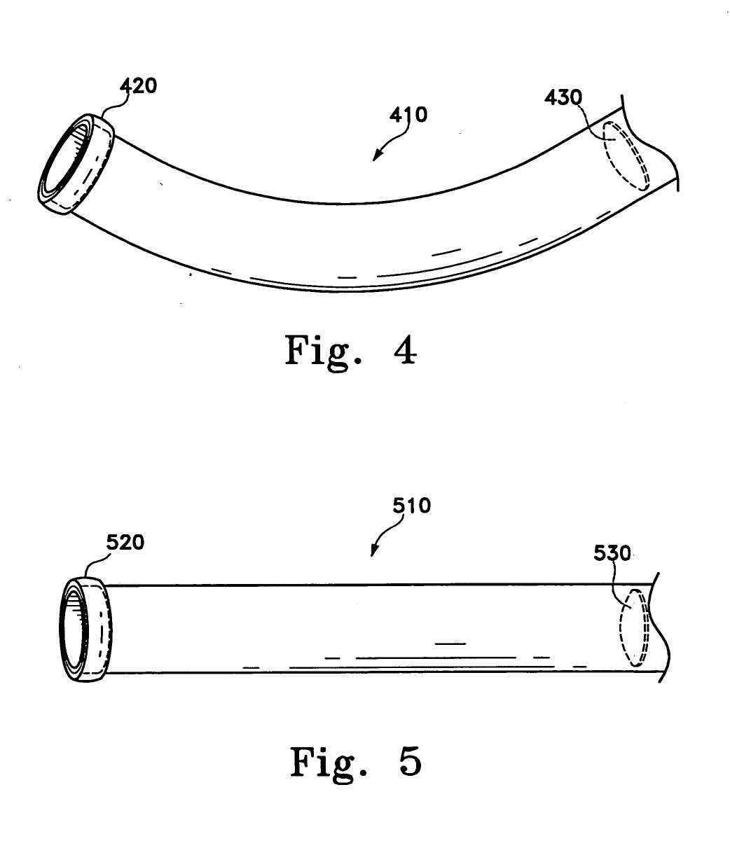

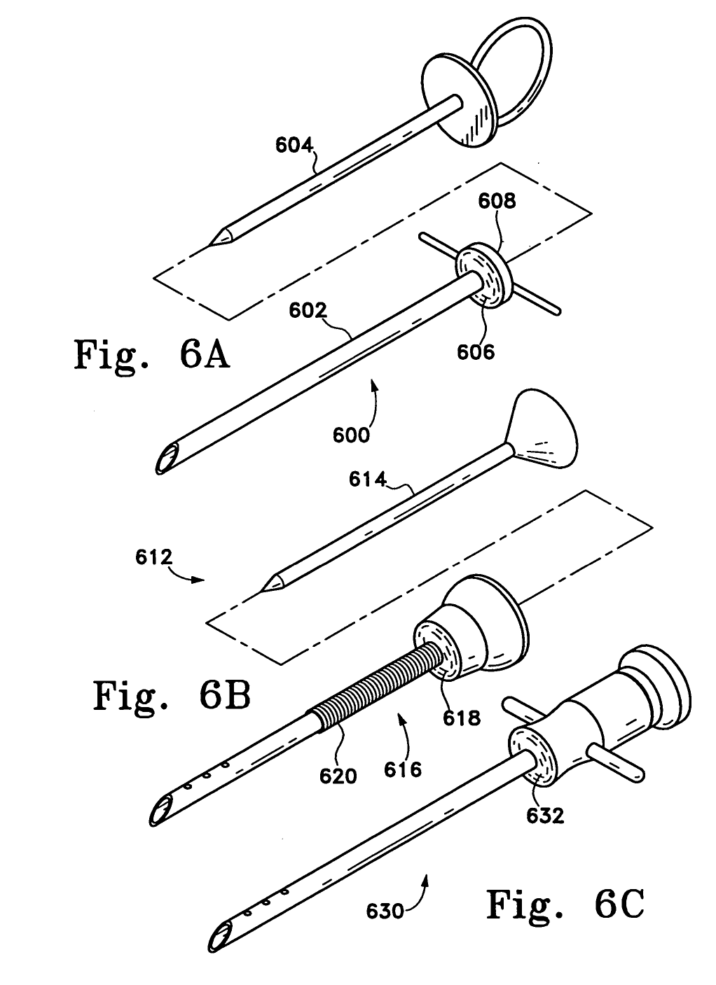

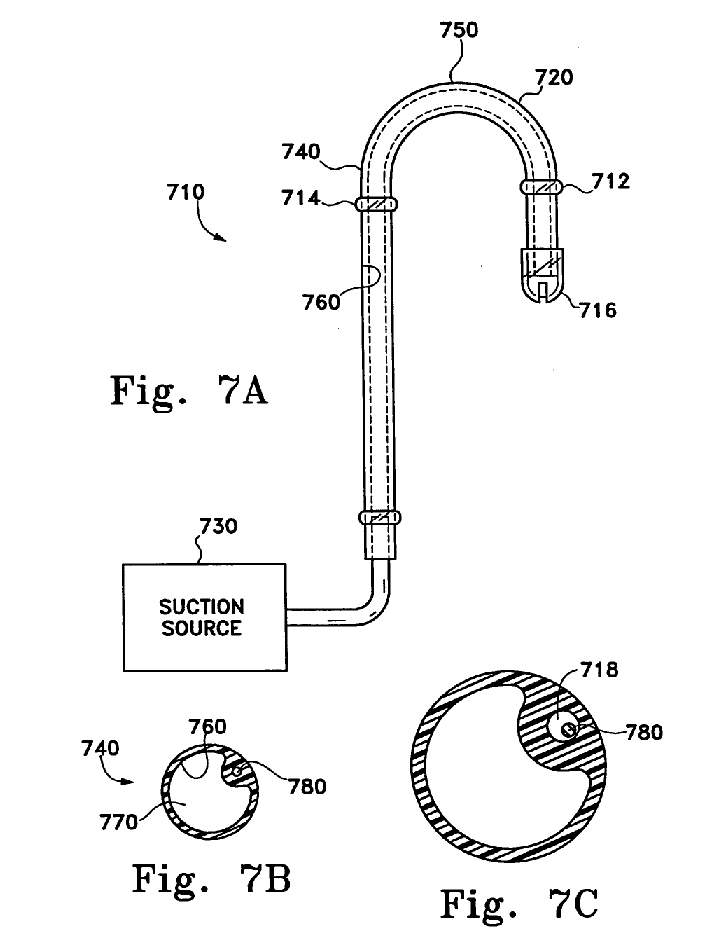

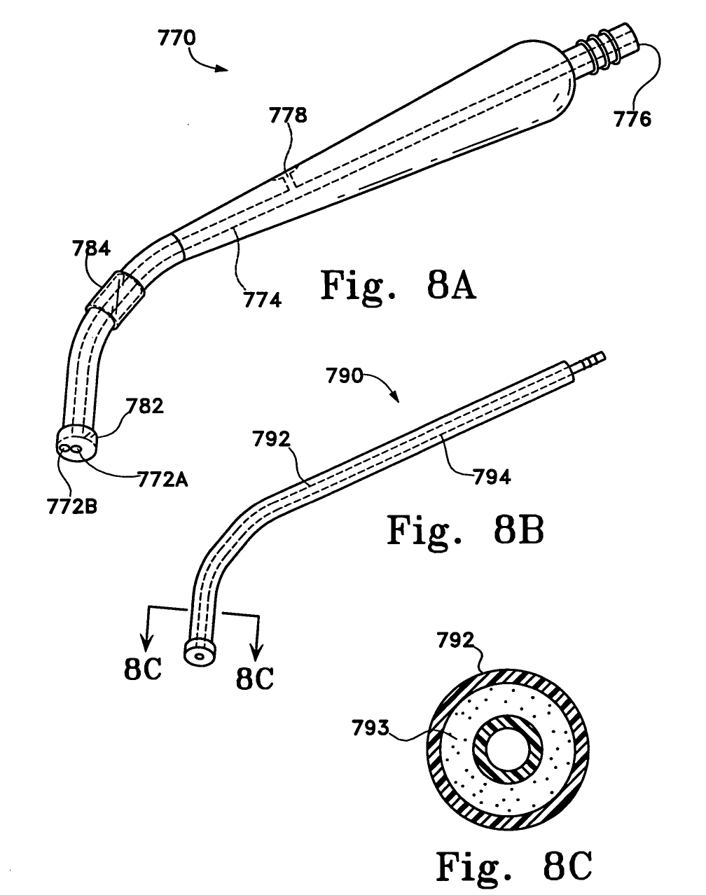

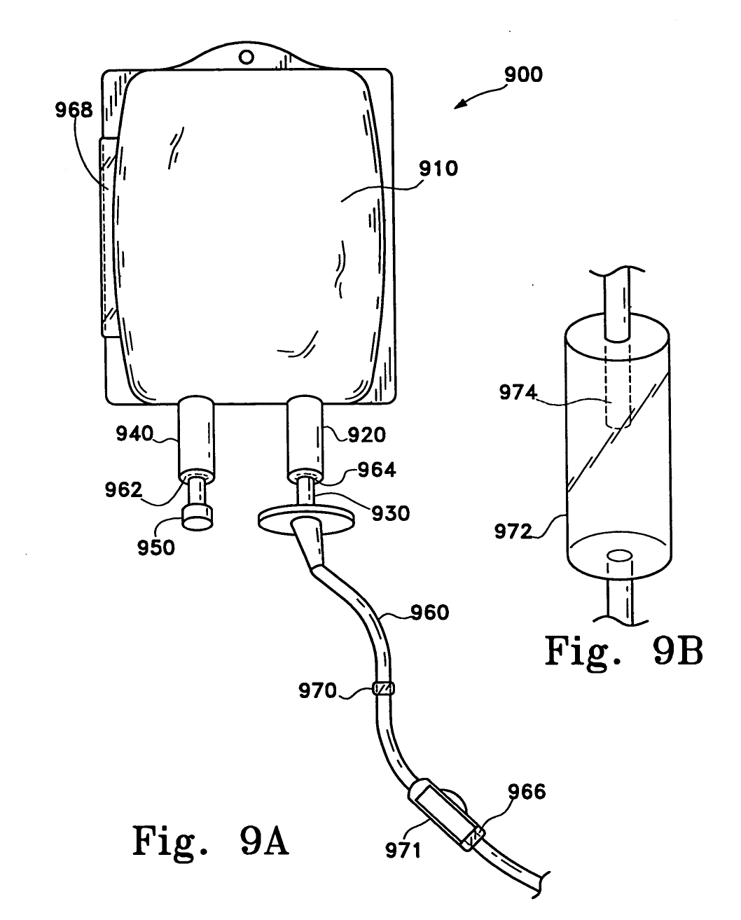

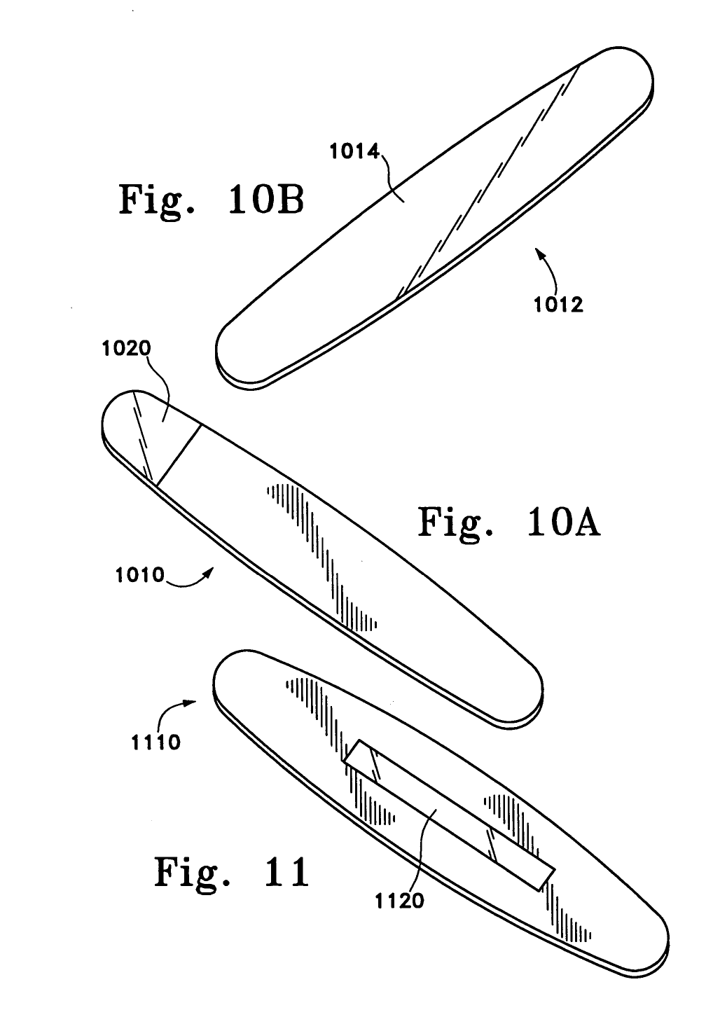

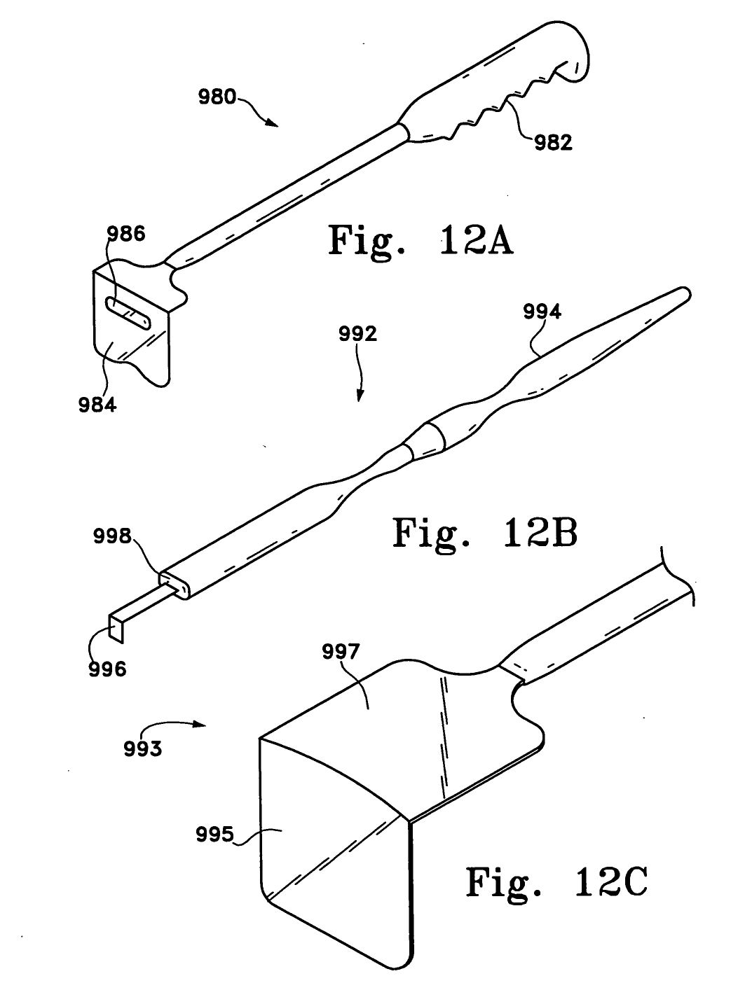

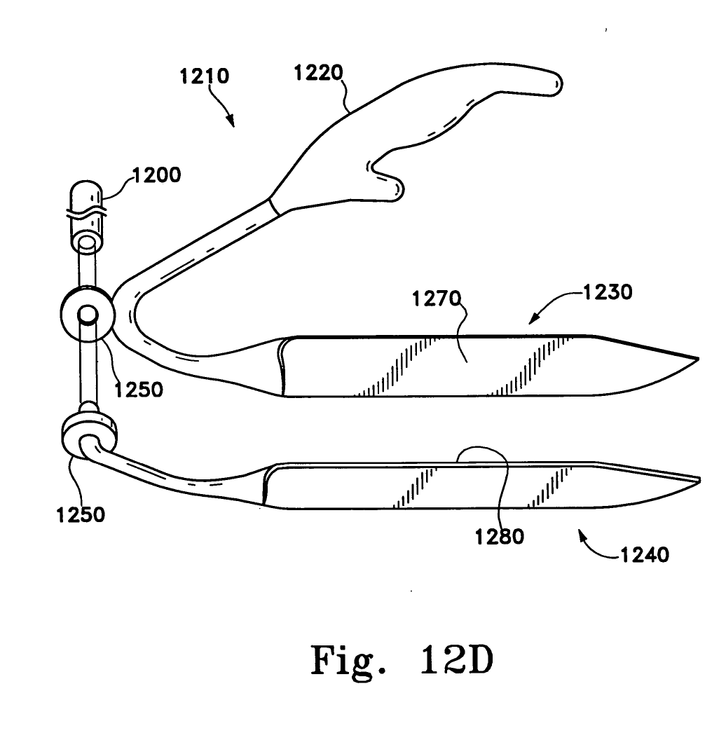

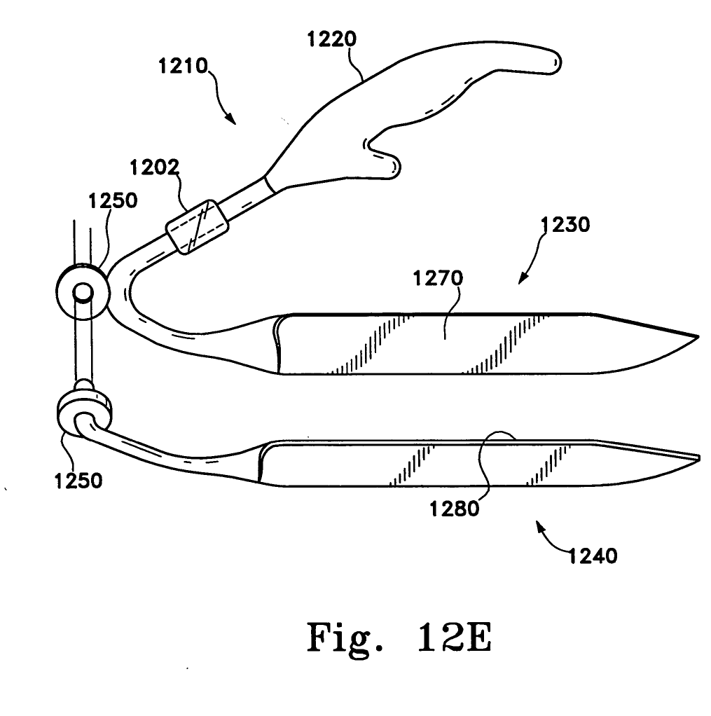

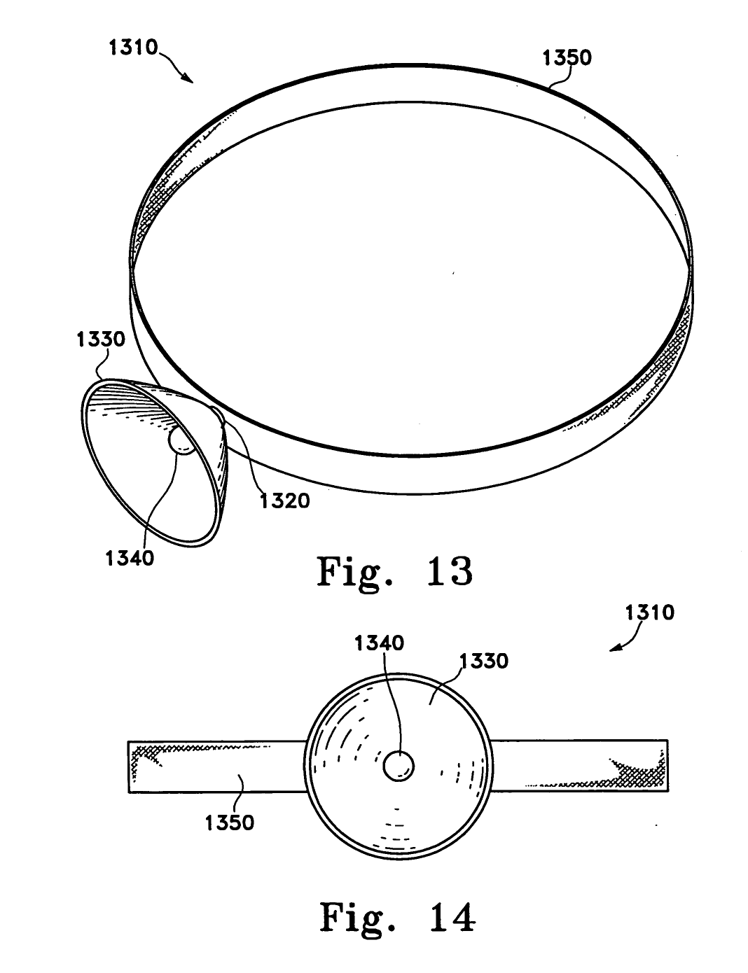

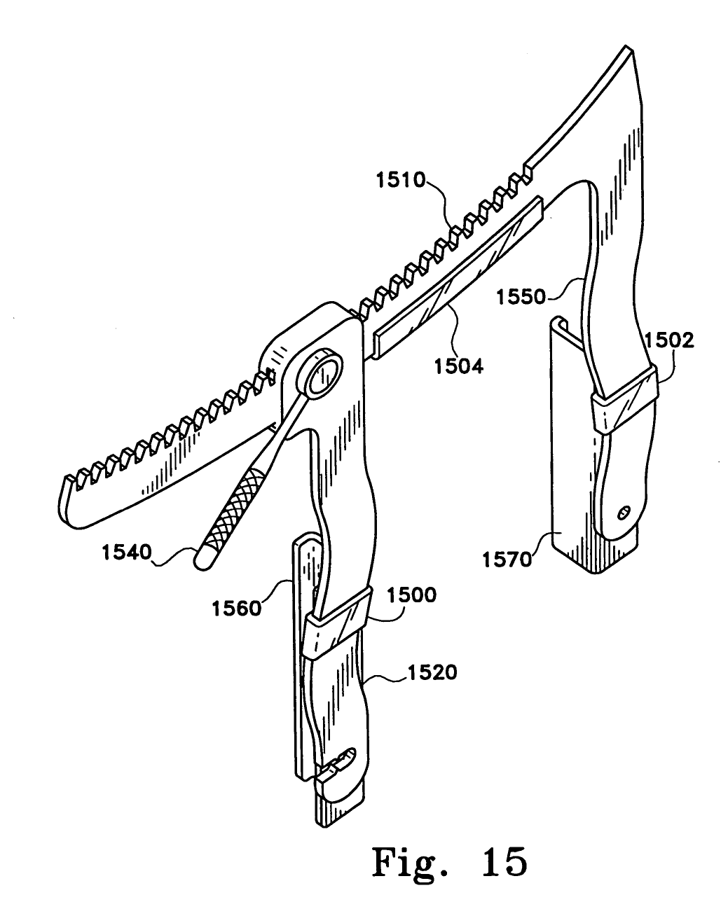

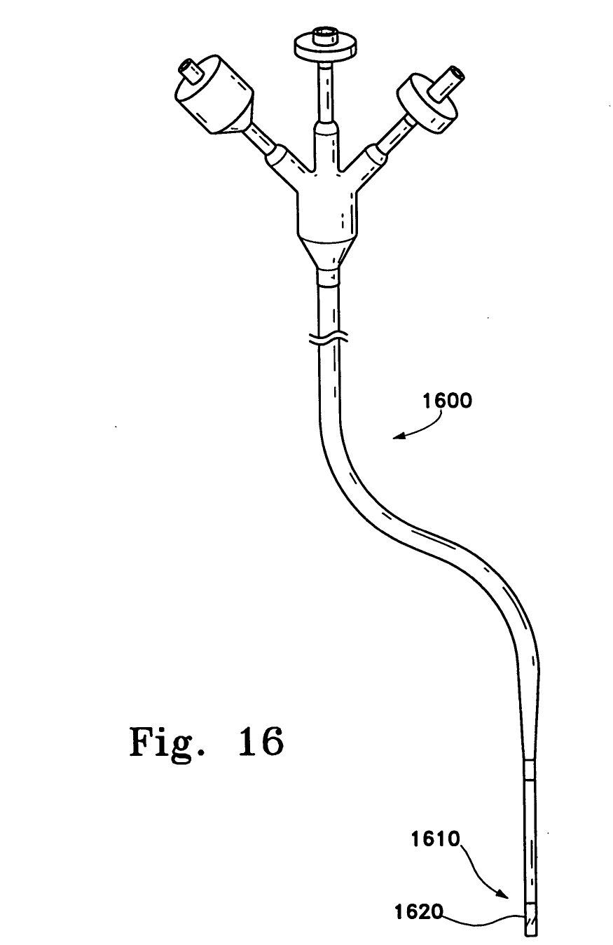

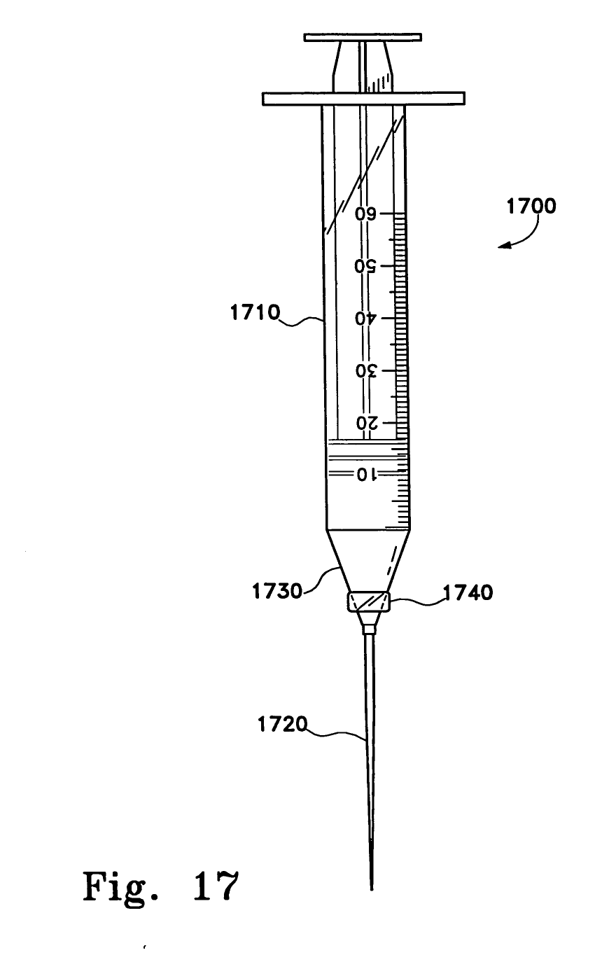

A dental suction appliance is shown in FIG. 7A. The appliance 710 may include a suction tube component 720 that is connected to a suction source 730. The suction source 730 may be a standard dental suction apparatus that will be known to those skilled in the art. The suction tube component 720 includes a lightweight, flexible, elongate tubular member 740 that may comprise a translucent or light-conducting polymeric material. The tubular member 740 may be curved to include a hook 750.

The elongate tubular member may have a central bore 770 and a tubular wall 760 that may carry a bendable shaping element 780 (allowing the tubular member to be curved or hooked), all as displayed in FIG. 7B. The one or more chemiluminescent regions 712 (near the distal end of the device), 714 (intermediate the shaft 710), 716 (at the distal-most end of the device as placed into the mouth) may be positioned as desired during design and manufacturing, if fixed, or as adjusted if designed in such a way. As is shown in FIG. 7C, the region surrounding the shapeable element 780 may be enlarged to serve as a chemiluminescent chamber 718. Indeed, in many variations of the dental suction appliances, the stiffener member is not an independent member, the designers choosing to utilize a tubing that is plastic or formable by hand. Such a chamber may be used as a longitudinal chemiluminescent chamber 718.

The one or more chemiluminescent regions may be positioned so that the mouth is illuminated when the suction is effected.

Surgical Suction Appliance

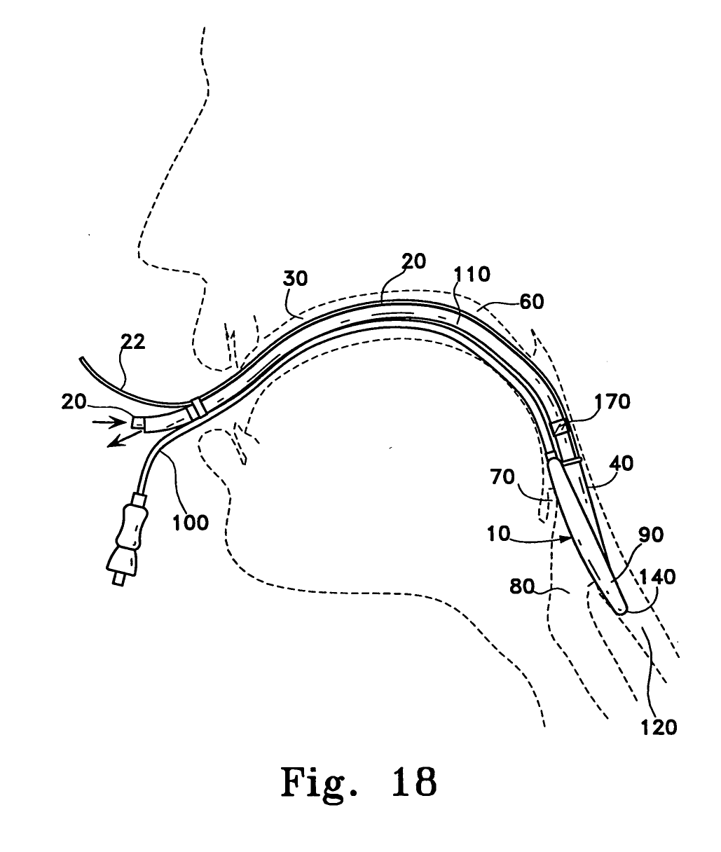

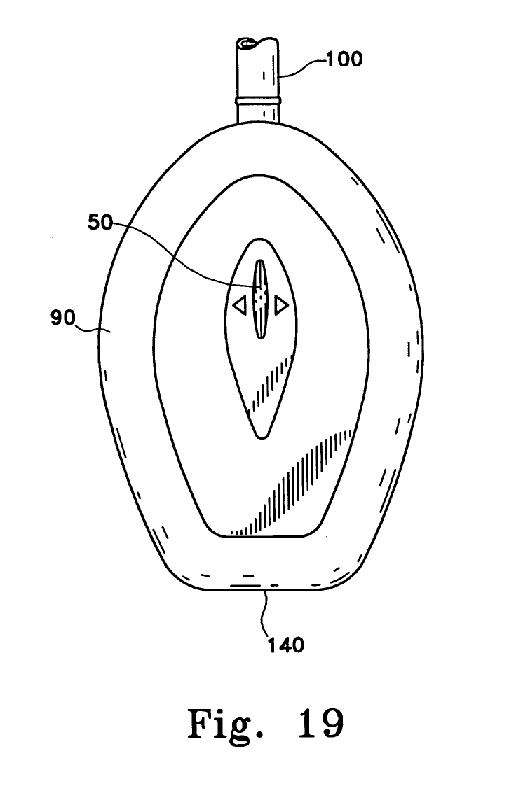

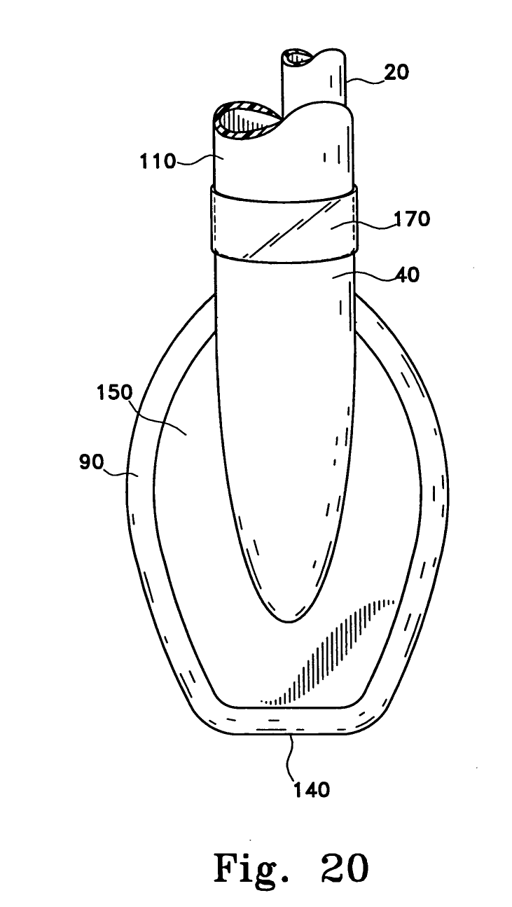

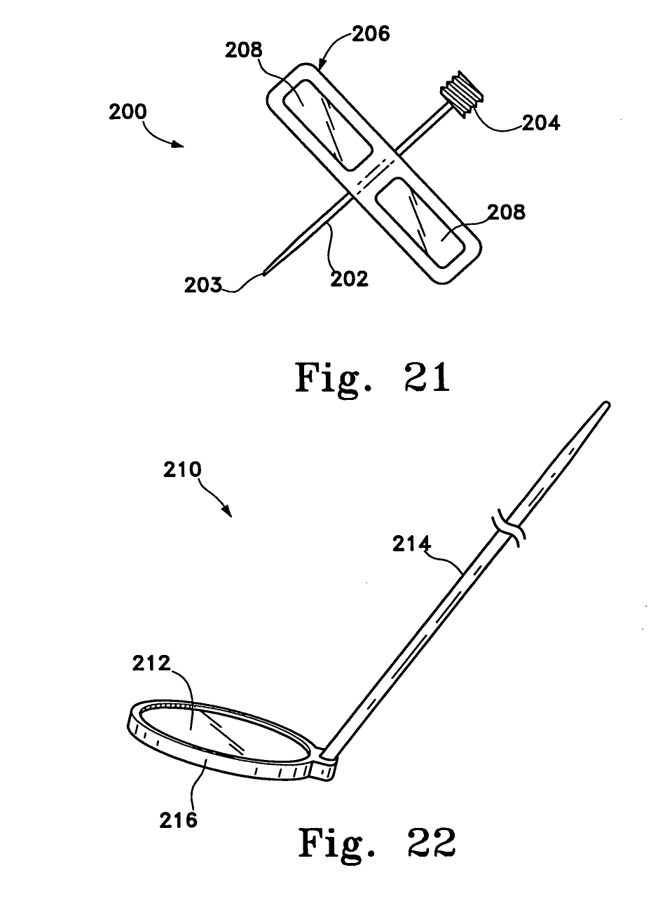

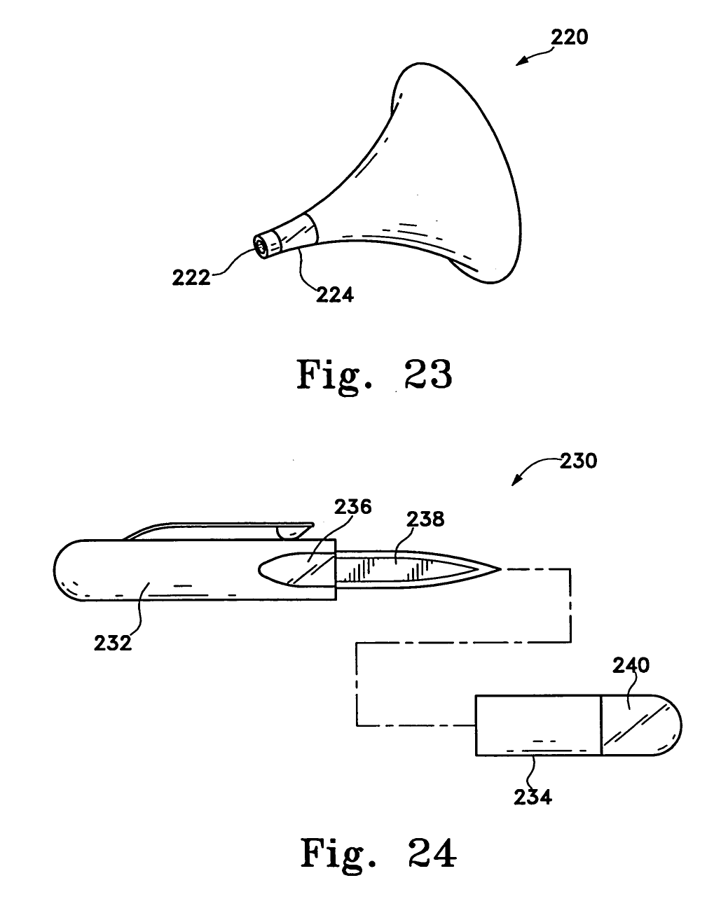

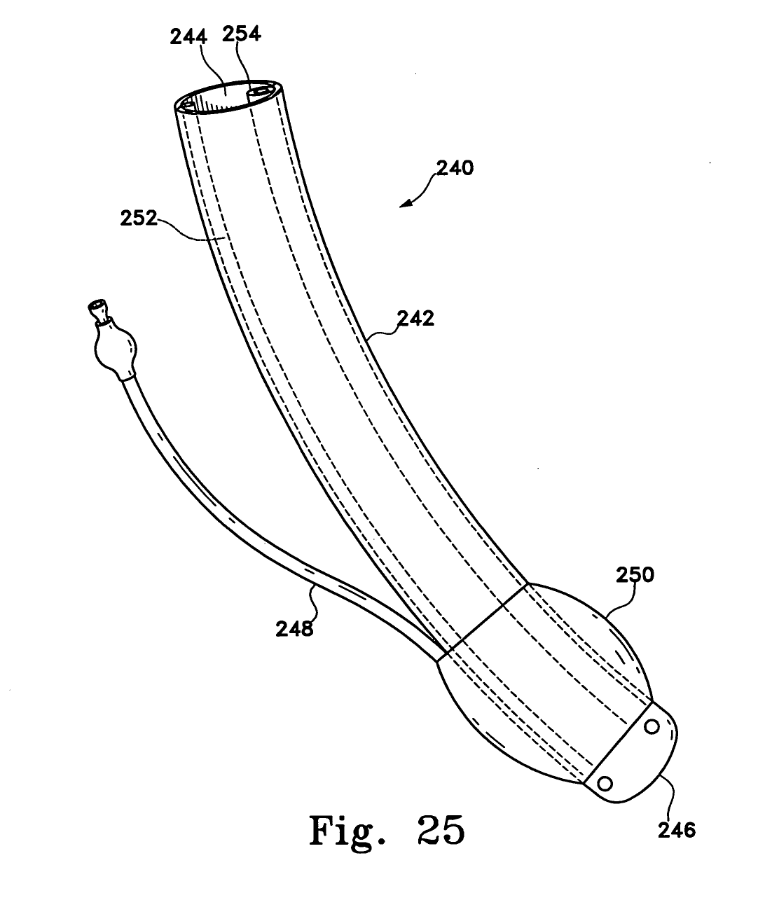

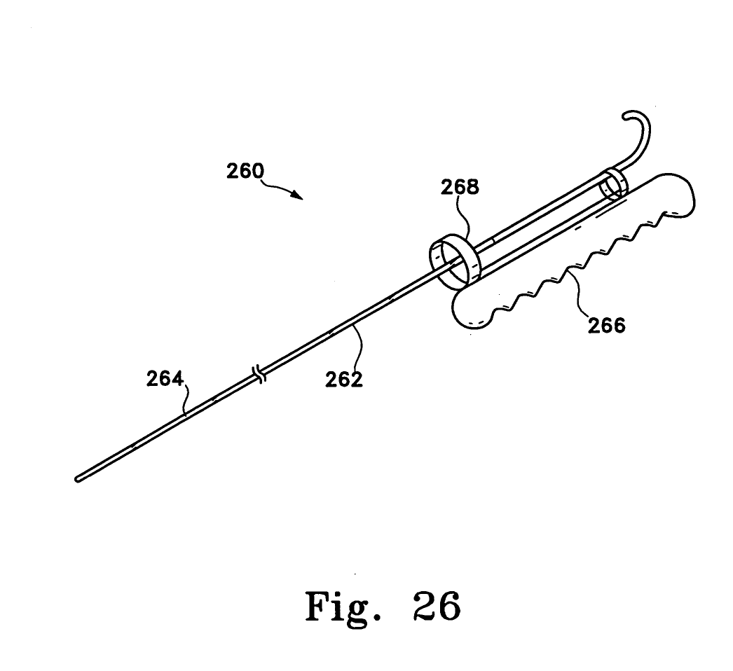

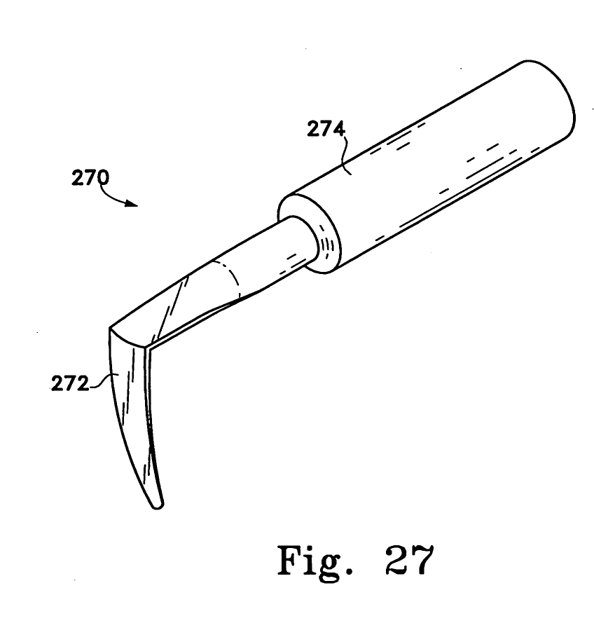

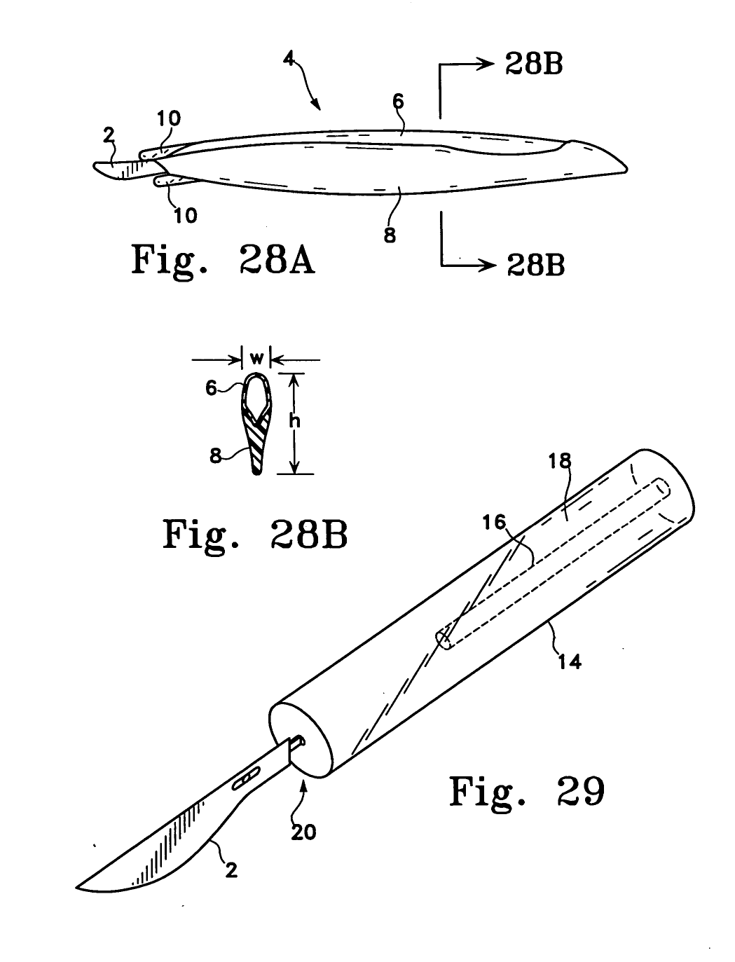

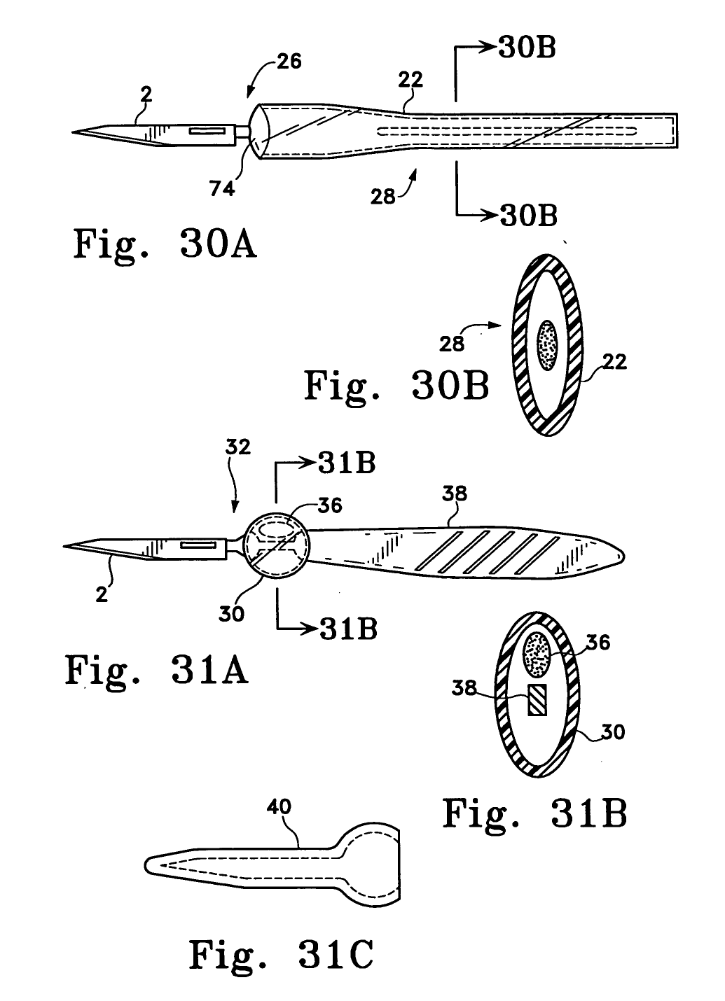

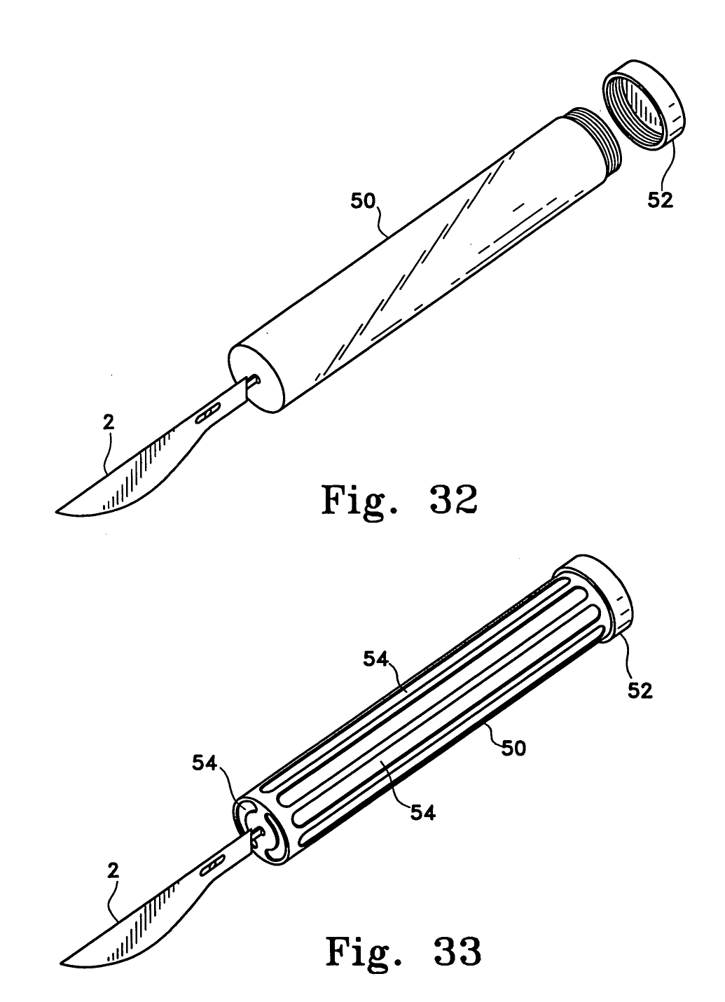

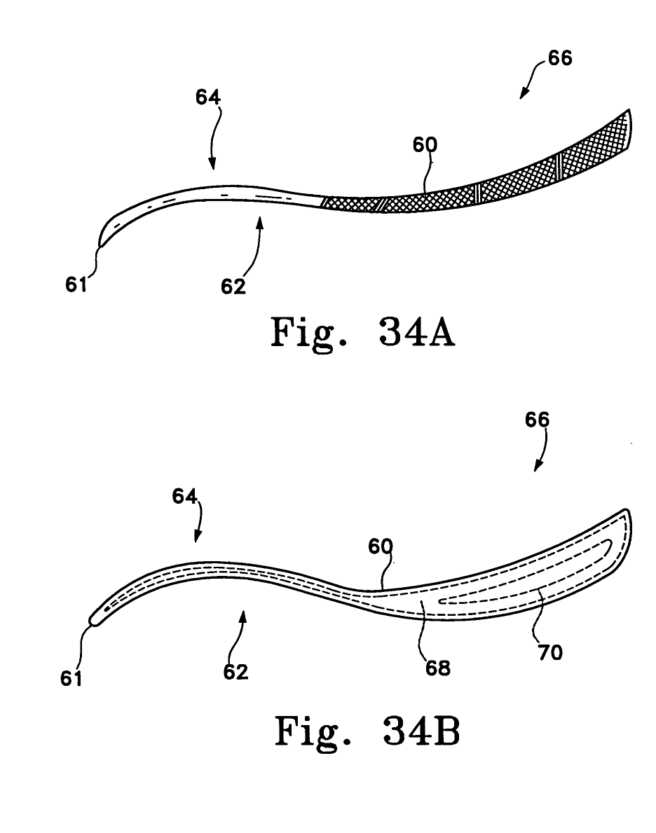

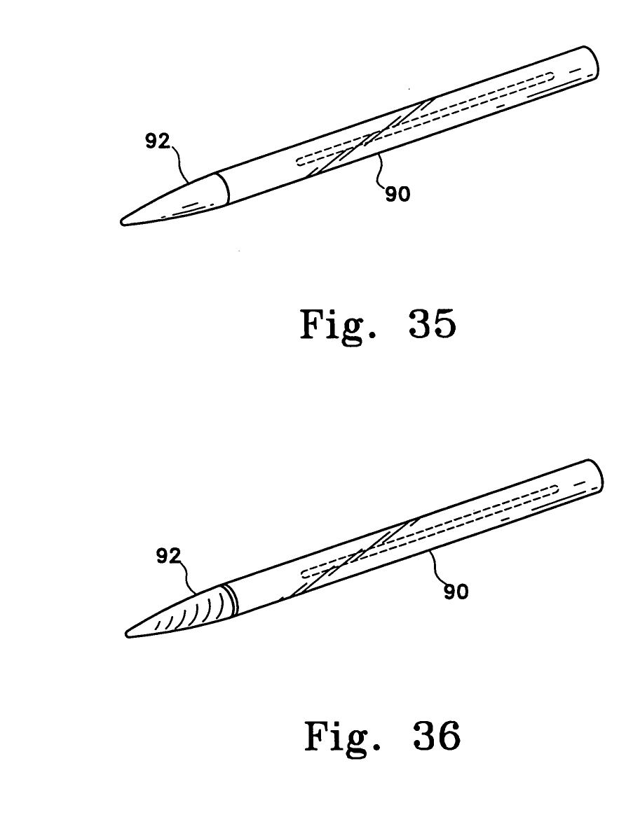

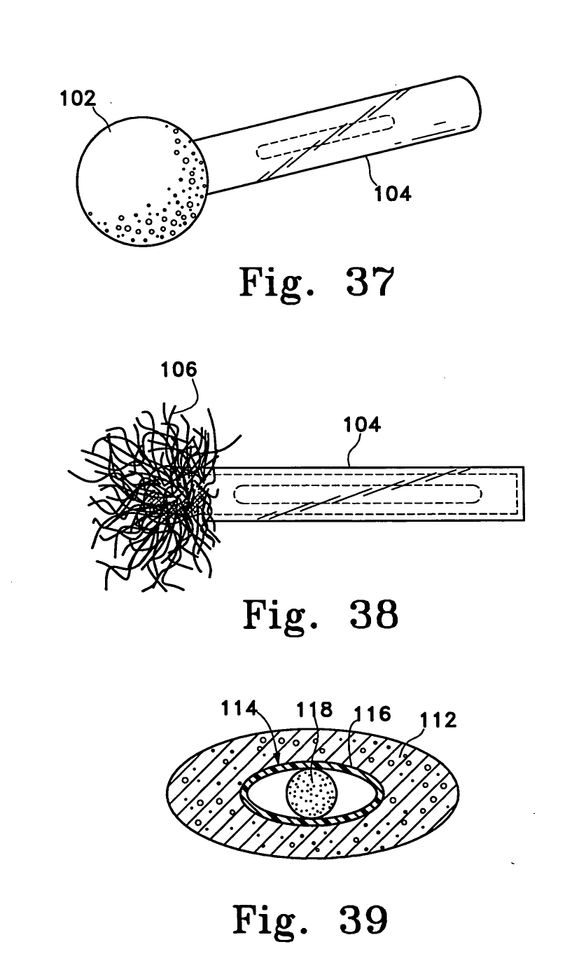

As shown in FIG. 8A, a surgical suction appliance 770 is similar in structure and may be similarly made. Surgical suction appliance 770 includes one or more suction ports 772A, 772B, a lumen 774 connecting that port to a suction source (not shown) typically connected to proximal opening 776, and a suction-breaker port 778 allowing the user to break the vacuum to the distal end port when necessary. Placement of the chemiluminescent regions within or upon such a surgical device is also similar to that described above. Chemiluminescent source 782 is shown in FIG. 8A at the distal end of the device near the suction port 772. Another chemiluminescent source 784 is shown farther up the shaft of the device.

FIG. 8B depicts another suction device, generally either for use in surgery or for dental use or any other procedure where fluids and substances are to be removed. The surgical device 790 in this variation includes a shaft 792 having a chemiluminescent light source 794 that is coextensive with shaft 792.

One example of a chemiluminescent source that is coextensive with the appliance shaft is illustrated in FIG. 8C where an annular space 793 between in inner and outer tubular member contains the chemiluminescent materials. Also, an elongated tube or member may run along the shaft that is not an annular volume nor in a coaxial relationship with the main lumen providing the suction. The chemiluminescent reservoir or source may run side by side with the suction lumen. Still, as with many of the devices disclosed herein, the chemiluminescent source or region may be disposed in a number of different locations along the medical device.

Surgical suction devices may further include a number of ancillary medical devices, such as detachable mosquito clamps, staplers, and electrocautery probes. The electrocautery probe may include a distal resection electrode that is mounted between a pair of arms. The arms are joined at their proximal ends to an electrode lead that is coupled via a handle to a source of electrocautery current. This structure allows practice of a resection procedure that involves connecting a cauterizing voltage to the electrode and moving the electrode slowly through or over a tissue. Via the energy applied through the electrode, the tissue in contact with that electrode is excised. Chemiluminescent light sources added in the vicinity of the probe for lighting of the excision do not interfere with the electrocautery devices nor does the electrocautery probe interfere with the chemiluminescent illumination of the site.

Additionally, an LED may be positioned at the distal end of the device to illuminate the surgical field. A wire(s) supplying voltage to the LED may run along the tubular member of the suction device.

1-67. (cancelled)

68. A suction appliance comprising:
a tube formed from a light-conducting polymer and having
a proximal end for mating to a suction source,
a distal end, and
a tubular wall including a first void at least partially filled with a chemiluminescent material, the tubular wall forming a channel for transmitting suction from the proximal to the distal end.

69. The suction appliance of claim 68, wherein the tube further includes a shapeable element disposed longitudinally with at least a portion of the tubular wall.

70. The suction appliance of claim 68, wherein the tube further includes a second void and a third void at least partially filled with a chemiluminescent material.

71. The suction appliance of claim 69, wherein the shapeable element is embedded in the tubular wall.

72. The suction appliance of claim 68, further comprising a shapeable element disposed within the void, and wherein a volume of the void formed by the tubular wall is filled at least partially by the shapeable element, and at least partially by the chemiluminescent material.

73. The suction appliance of claim 68, wherein the polymer is a shapeable polymer.

74. A suction appliance, comprising:
a first tube formed from a light-conducting polymer and having
a proximal end adapted to mate with a suction source,
a distal end, and
a tubular wall forming a channel for transmitting suction from the proximal end to the distal end; and
a closed container disposed with a least a portion of the first tube and containing a chemiluminescent material.

75. The suction appliance of claim 74, wherein the closed container is tubular.

76. The suction appliance of claim 75, wherein the tubular closed container is disposed within the first tube.

77. The suction appliance of claim 76, wherein the tubular closed container is coaxially disposed within the first tube.

78. The suction appliance of claim 74, further comprising a second and a third closed container, and wherein the first chemiluminescent container is disposed proximate to the proximal end of the tube, and the second chemiluminescent container is disposed proximate to the distal end of the tube.

79. A suction appliance comprising:
a first tube formed from a light-conducting polymer, the first tube having proximal and distal ends and a tubular wall;
a second tube disposed at least partially within the first tube, and having a proximal end adapted to attach to a suction source, a distal end, and a tubular wall, the tubular wall of the first tube coupled to the tubular wall of the second tube, thereby forming a void, the void at least partially filled with chemiluminescent material.

80. The suction appliance of claim 79, wherein the second tube is formed from a shapeable material.

81. A method, comprising:
activating a chemiluminescent material contained within a first void of a tube, the tube having proximal and distal ends;
connecting the proximal end to a suction source; and
suctioning with the distal end while illuminating a surface.

82. The method of claim 81, further comprising:
activating a chemiluminescent material contained within a second void of the tube.

83. The method of claim 81, further comprising:
shaping the tube to select the surface being illuminated.

84. A method, comprising:
activating a chemiluminescent material in a container;
attaching the container to a tube having a proximal end, a distal end, and a channel from the proximal to the distal end for transmitting suction from the proximal to the distal end;
providing a suction to the proximal end of the tube; and
suctioning with the distal end of the tube while illuminating a surface with light emitted from the chemiluminescent material.

85. The method of claim 84, further comprising:
shaping the tube to select the surface for illumination.

86. The method of claim 84, further comprising:
activating a chemiluminescent material in a second container;
attaching the second container to the tube;
illuminating an other surface with the chemiluminescent material in the second container.

87. The method of claim 86, further comprising:
shaping the tube to select the other surface for illumination.
